(19) United States
(12) Patent Application Publication
(10) Pub. No.: US 2004/0076303 A1
(43) Pub. Date: Apr. 22, 2004

(54) Multimedia adapter to an acoustic stethoscope

(76) Inventors: Andrey Vyshedskly, Boston, MA (US); William Kania, Westborough, MA (US); Raymond Murphy, Wellesley, MA (US)

Correspondence Address: Andrey Vyshedskly, Suite 4990, 1153 Centre St., Boston, MA, 02130, US

(21) Appl. No.: 10/670,466

(22) Filed: Sep. 26, 2003

Related U.S. Application Data
(60) Provisional application No. 60/414,395, filed on Sep. 30, 2002.

(51) Int. Cl. A61B 7/04

(52) U.S. Cl. 381/67

(57) ABSTRACT

The invention herein discloses a multimedia acoustic stethoscope that has look and feel of a conventional acoustic stethoscope but allows a medical practitioner to transmit sounds from stethoscope to a recording device, and to transmit recorded sound back to the stethoscope for playback. The recording device could be a tape recorder, a digital recorder, a personal computer, a pocket PC, a handheld PC, a tablet PC, or a PDA. The data can be transmitted via a wire or wirelessly, for example using Bluetooth wireless technology.

Priority is claimed by provisional application No. 60/414,395 filed on Sep. 30, 2002 entitled Multimedia adapter to an acoustic stethoscope for sounds recording and playback.

### FIELD OF THE INVENTION

The invention relates generally to stethoscopes, more particularly to multimedia stethoscopes.

### BACKGROUND OF THE INVENTION

Stethoscopes are widely used by medical personnel to listen to body sounds. Unfortunately stethoscopes do not allow recording, playback, amplification, filtering, or visualization of these sounds. Improvements to stethoscope are well known. U.S. Pat. No. 6,533,736 to Mark Moore discloses a wireless stethoscopic apparatus comprised of an auscultation piece removably secured to a hearing piece. Housed within the auscultation piece is a conventional radio frequency chip including a microphone that will enable transmission of radio frequency without the use of wires. A transmission system is housed within the auscultation piece, while the hearing piece includes a link manager receiver device for receiving the radio signals wirelessly from the transmission system and enabling sound to be heard via the hearing device from the auscultation piece. This apparatus does not allow recording, playback, or visualization of sounds.

U.S. Pat. No. 5,932,849 to Alan P. Dieken discloses a stethoscope chest piece having an acoustic-to-electrical transducer residing within the acoustic pathway of the chest piece. The transducer resides within a mounting that can provide shock attenuation and vibration isolation. The transducer preferably resides within a coaxial position in the acoustic pathway. The chest piece is useful in a stethoscope which can be electrically connected to auscultation systems. Although this apparatus allows sound recording, it does not allow playback of sound.

U.S. Pat. Nos. 5,844,995 and 5,717,769 to Christopher A Williams disclose adapters and methods for reversibly converting a standard stethoscope to a multimedia and/or telemedicine ready stethoscope. The adapter includes a housing having a conduit for conducting sound therethrough, a first coupler for coupling the housing to the transducer of a standard stethoscope, a second coupler for coupling the housing to the sound conduction hose of a standard stethoscope, and a second transducer for converting an audible tone to an electronic signal. Although this apparatus allows sound recording, it does not allow playback of sound.

U.S. Pat. No. 5,213,108 to Mark S Bredesen discloses an visual display stethoscope for use in the auscultation of body sounds is disclosed. The stethoscope is adapted for display, manipulation and analysis of the received body sounds. The disclosed invention includes a stethoscope electronically coupled to a display module. The display module has the ability to display an analog representation of the received body sounds and includes menu keys for selecting among the various functions provided by the stethoscope for manipulation and analysis of the waveform data. These functions include real time analog filtering of displayed waveforms, digital filtering of stored waveforms, and interval timing between strategic positions in the body sound waveforms. Although this apparatus allows sound recording and visualization, it does not allow playback of sound.

Electronic stethoscopes are also well known. Normally electronic stethoscopes have a microphone, an electronic circuit for sound amplification and filtering, and an internal speaker. The U.S. Pat. No. 5,347,583 to Dieken, et al. discloses a binaural electronic stethoscope adapted to receive auscultatory sounds from a body and adapted to transmit the auscultatory sounds to a user.

Although electronic stethoscopes have been available for over 10 years the medical community was slow to accept them. The possible reasons include lack of clear benefits, high cost, and dependence on the battery.

### BRIEF SUMMARY OF THE INVENTION

The invention herein disclose a multimedia acoustic stethoscope that has the look and feel of a conventional acoustic stethoscope but allows a medical practitioner to transmit sounds from the stethoscope to a recording device, and to transmit the recorded sound back to the stethoscope for playback. The recording device can be a tape recorder, a digital recorder, a personal computer, a pocket PC, a handheld PC, a tablet PC, or a PDA. The data can be transmitted via a wire or wirelessly. Bluetooth technology is an example of the wireless technology that can be used.

The disclosed multimedia stethoscope has the following advantages:

1. The multimedia stethoscope allows recording of the sound into any external or internal recording device.

2. The multimedia stethoscope allows playback of the prerecorded sounds from any external or internal recording devices.

3. The cost of the multimedia stethoscope is reduced compared with a recording electronic stethoscope.

4. It is more convenient for the medical practitioner to continue using the acoustic stethoscope that he/she is accustomed to use.

5. The multimedia stethoscope does not depend on the battery for the auscultation process.

6. Played back sound can be amplified and filtered by the recording device.

The particular advantages of the playback function of the multimedia stethoscope can be illustrated by the following example. When the medical practitioner is using a ‘recording only’ stethoscope with a PDA to record sounds, he/she needs to disconnect the recording stethoscope and connect headphones in order to play the sound back. The headset has to be used since internal PDA speakers are not intended for reproduction of low frequency sounds such as heart sounds. Note that the headset has to be carried along with PDA and stethoscope. The multimedia stethoscope disclosed herein would be used for both recording and playback eliminating the need for headphones.

### BRIEF DESCRIPTION OF THE DRAWING

FIG. 1 is an overall design of the multimedia stethoscope.

FIG. 2 is an overall design of the multimedia adapter for converting an acoustic stethoscope into a multimedia stethoscope.

### DETAILED DESCRIPTION OF THE INVENTION

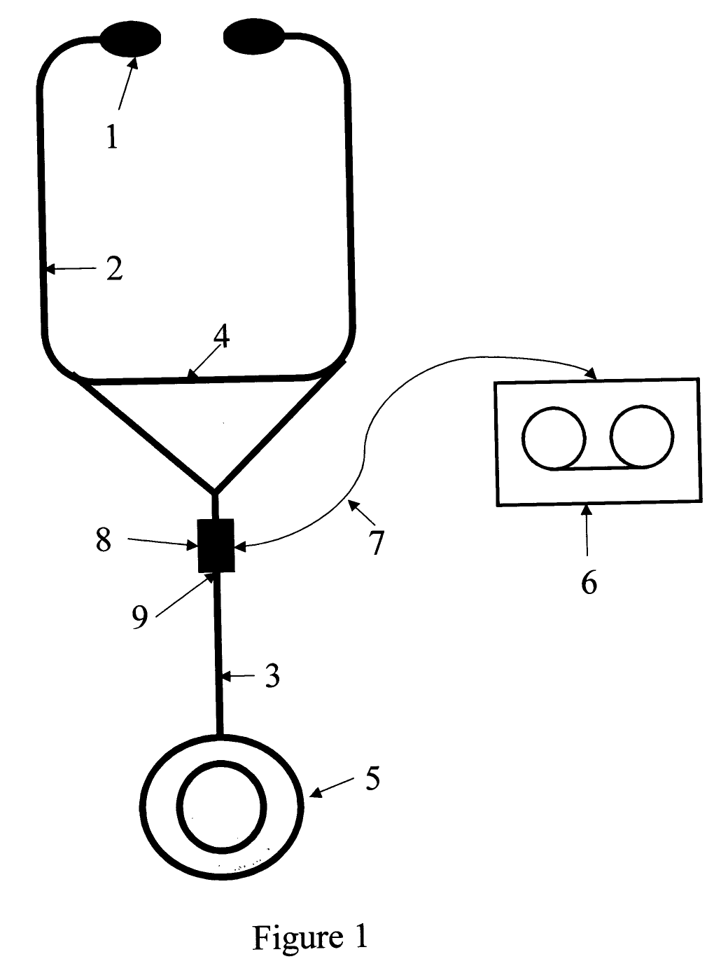

FIG. 1 shows an overall design of the multimedia stethoscope. The multimedia stethoscope consists of the following elements: ear tips 1, binaural attachment 2, tubing 3, spring 4, and chest piece 5. The multimedia adapter 8 houses a microphone and a speaker. The acoustic vibrations in the stethoscope tubing 3 are converted into an electrical signal in the multimedia adapter 8 and transmitted via a wire or wirelessly 7 to a recording device 6. The sound for playback is transmitted from the recording device 6 via a wire or wirelessly 7 to the multimedia adapter 8. The sound played back by the speaker located in the multimedia adapter 8 sends acoustical vibrations to ear tips 1 via the tubing 3.

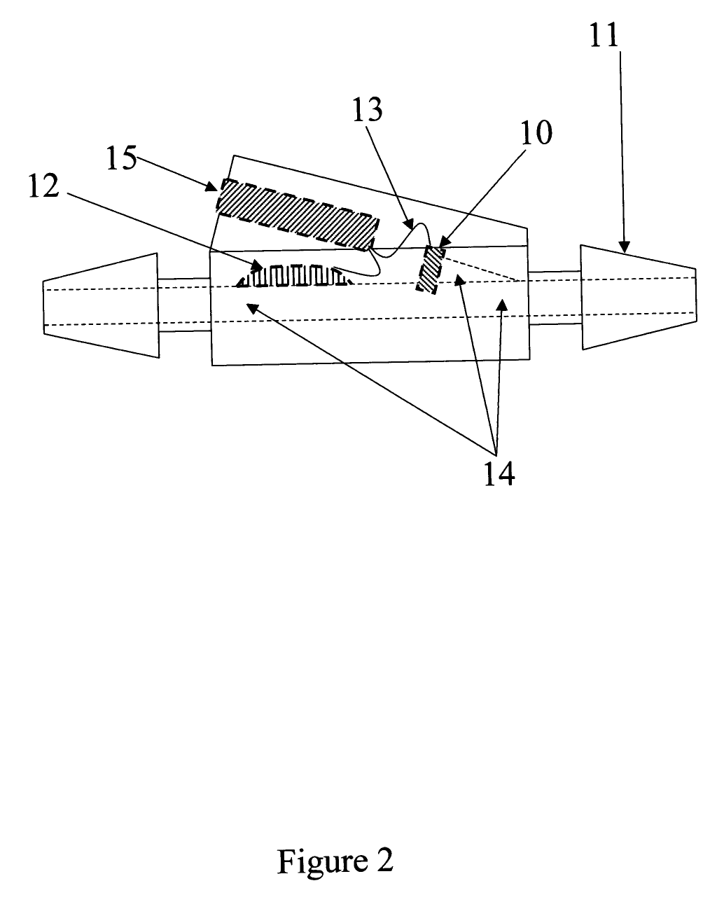

FIG. 2 is a preferred embodiment of the multimedia adapter. The multimedia adapter consists of a housing 8 holding a microphone 10, the speaker 12 and an audio jack or wireless signal transmitter 15. The suitable microphone for the sound pick-up can be omnidirectional electret microphone. The suitable speaker for sound playback need to have good frequency response down to 20 Hz. The microphone 11 facing the air canal 14 is used to record sound heard by the user. This sound signal is then transmitted via the wired audio jack or wireless signal transmitter 15 to the recording device. The recording device can then play the sound back via the speaker 12 that faces the air canal 14. The playback sound arrives to the user via the binaural attachment 2. The multimedia adapter is connected to the tubing 3 via a barbed connector 11. The suitable acoustic stethoscope is Littman Classic II.

The recording device can be a PDA such as Compaq iPAQ5450 Pocket PC. The signal from the microphone 10 is transmitted to the PDA's microphone input port. The transmission can be via the wire connected to an external 3.5 mm microphone jack or wirelessly via bluetooth headset protocol. No modification or special hardware is required with iPAQ5450. The PDA can display the waveform of the audio signal, called phonocardiogram, on its screen and store the data for later retrieval, transfer, or playback. Also, the PDA can be programmed to perform the automatic analysis of the acoustic signal.

We claim:

1. A multimedia adapter to an acoustic stethoscope comprised of:
(a) a microphone mounted in the chest piece or tubing for conversion of sounds heard by an operator into an electrical signal and transmission of said electrical signal to a recording device,
(b) a speaker mounted in the chest piece or tubing for playback of sounds from the recording device,
(c) a wired or wireless connector from said microphone and said speaker to the recording device, and
(d) means to hold said microphone, said speaker, and said connector within said acoustic stethoscope,
whereby said multimedia adapter will enable said acoustic stethoscope to be used for sound recording and playback.

2. The multimedia adapter of claim 1 wherein said microphone, speaker, and connector are mounted inside a chest piece attached to said acoustic stethoscope.

3. The multimedia adapter of claim 1 wherein said microphone, speaker, and connector are mounted inside a housing incorporated into the tubing of said acoustic stethoscope.

4. The microphone of claim 1 selected from a group consisting of a condenser microphone, an electret microphone, and an accelerometer.

5. The wireless connector of claim 1 wherein wireless protocol selected from a group consisting of a bluetooth protocol and a Wi-Fi protocol.

6. The bluetooth protocol of claim 5 wherein a headset profile is used to transmit data to and from the recording device.

7. The multimedia adapter of claim 1 wirelessly connected to the recording device wherein an on/off button located on said multimedia adapter is used to turn wireless connection to said recording device on and off.

8. The recording device of claim 1 selected from a group consisting of a tape recorder, a digital recorder, a personal computer, a PDA, a handheld computer, and a tablet PC.

9. The recording device of claim 1 visualizing the sound in both time and frequency domains.

10. The recording device of claim 1 analyzing normal and abnormal sounds.

11. The recording device of claim 1 playing back amplified and filtered sound.
